(19) United States
(12) Patent Application Publication
(10) Pub. No.: US 2009/0252675 A1
(43) Pub. Date: Oct. 8, 2009

(54) Radionuclide Therapy for Urinary Bladder Cancer

(76) Inventors: R. Keith Frank, Lake Jackson, TX (US); Daniel J. Macey, Birmingham, AL (US); Jaime Simon, Angleton, TX (US); Alan D. Strickland, Lake Jackson, TX (US)

Correspondence Address: TECHNOLOGY LAW, PLLC, 3595 N. SUNSET WAY, SANFORD, MI, 48657, US

(21) Appl. No.: 12/225,464

(22) PCT Filed: Mar. 23, 2007
(86) PCT No.: PCT/US2007/007392

§ 371 Date: Sep. 22, 2008

Related U.S. Application Data
(60) Provisional application No. 60/786,991, filed on Mar. 29, 2006.

(51) Int. Cl. A61K 51/04; A61P 39/04; A61P 35/00

(52) U.S. Cl. 424/1.65

(57) ABSTRACT

The compounds used in this method for the treatment of bladder cancer comprise a radioisotope particle emitter, such as alpha- or beta-emitters or low energy gamma-emitters, associated with a chelating agent (for metallic radioisotopes) or other compound (by covalent bond for iodine or other non-metals) selected such that these permit delivery of the radionuclide by an I.V. injection that is then cleared into the bladder by the kidneys. Furthermore, it is expected that these radioisotopes are formulated with customary pharmaceutically-acceptable salts, adjuvants, binders, desiccants, diluents, and excipients.

### FIELD OF THE INVENTION

The present invention concerns treatment of urinary bladder cancer in animals, including humans, by use of radionuclide therapy.

### BACKGROUND OF THE INVENTION

Cancer of the urinary bladder was diagnosed in over 63,000 people in the United States in 2005, accounting for 7% of all malignancies in men and 4% in women (American Cancer Society, Cancer Facts & Figures, 2005). There were about 13,000 deaths from cancer of the urinary bladder in 2005 in the United States, accounting for 2.3% of the cancer deaths (American Cancer Society, Cancer Facts & Figures, 2005). The cost of care for treatment of a patient having bladder cancer from diagnosis to death is higher than any other cancer and in the United States generates $4 billion dollars in direct costs. However, these costs are reduced significantly when this cancer is detected early. As a result screening high risk groups by low cost testing methods to find this cancer early is desirable both for prognosis and costs. One method for such early testing is by using 22™ BladderChek™ Test by Matritech.

Cancer of the urinary bladder is graded primarily on the degree of invasion with superficial tumors being the lowest score, while muscle invasion and metastases are more serious. Cancer cell types also vary from carcinomas to leiomyosarcomas to small cell tumors. Mortality statistics worsen with the depth of invasion. Morbidity varies with both the depth of invasion and the type of treatment given.

The morbidity from cancer of the urinary bladder and the current treatment options are significant. Most bladder cancers are detected at an early stage as blood in urine is a reliable indicator to the patient to pursue the problem. The color of urine ranges from faintly rusty to deep red. Pain during urination can also be an indicator of bladder cancer as well as frequent urination and urgency. However, bladder tumors often cause no symptoms. Recently, it has been shown that bladder wall thickness measurements with CT imaging can provide improved diagnostic accuracy compared with the conventional cystoscopy.

Treatment for bladder cancer depends on a number of factors and these usually include how quickly the cancer is growing; and the number, size, and location of the tumors. Other factors are related to how far the cancer has spread to other organs/tissues in the body, the patient's age and general health. Most bladder cancers develop in the inside lining of the bladder. The mass often resembles a small mushroom attached to the bladder wall and may be diagnosed as a papillary tumor. Frequently more than one tumor is present.

The primary treatments available for cancer of the urinary bladder are radical cystectomy, radical radiotherapy, and chemotherapy, which chemotherapy is usually given in combination with either the surgery or the radiotherapy. Radical cystectomy is considered the “gold standard” for treatment, but there are several problems with the surgery [see, for example, Bassi, P, Curr. Opin. Urol. 10(5), 459-463 (2000); and Shipley, W. U., et al., Cancer 97(8 Suppl.), 2115-2119 (2003)]. Removal of the bladder requires diversion of the ureters, and the choice of diverting them to an incontinent ileal loop, a continent ileal loop, or a continent orthotopic device. The standard procedure is the incontinent ileal loop, which requires the patient to adjust to a life with constant seepage of urine onto the skin [see Zeitman, A. et al., Semin. Radiat. Oncol. 15(1), 55-59 (2005)]. Radical cystectomy, due to the dissection of pelvic nodes, usually results in nervous damage. Combined with the small bowel resection, this nerve damage to the pelvic gastrointestinal tract frequently results in intestinal morbidity. Urinary leakage, recurrent pyelonephritis, and intestinal obstruction are common problems [see Chahal, R. et al., Euro. Urol. 43(3), 246-257 (2003)]. The complication rate was between 25 and 35% of patients, regardless of age [see Clark, P. E., et al., Cancer 104(1), 36-43 (2005)]. Mortality in patients treated with radical cystectomy was 3.1% at 30 days, 8.3% at 3 months, and 63.5% at 5 years past the surgery [see Chahal, R. et al., Euro. Urol. 43(3), 246-257 (2003)].

The target volume in early stage bladder cancer is a thin layer of cancer cells that grow and spread in the superficial layers of the bladder wall. Intravesicular therapy with chemotherapeutic agents, Bacillus Calmette-Guérin (BCG), and/or Mitomycin C have been evaluated for superficial urinary bladder tumors [see, for example, Malmström, P. U. et al., Expert Rev. Anticancer Ther. 4(6), 1057-1067 (2004); Botteman, M. F. et al., Pharmacoeconomics 21(18), 1315-1330 (2003); Oosterlinck, W., Curr. Opin. Urol. 11(5), 511-515 (2001); and Oosterlinck, W., Minerva Urol. Nefrol. 56(1), 65-72 (2004)].

The chemotherapeutic agents can be given either as intravenous agents or intravesicular agents, but the concentration in the bladder is the important variable. At present, these therapies are considered adjuvant treatments for surgical control of the bladder cancer (typically localized, transurethral resections of tumor mass). One concern about localized, intravesicular therapy is the frequency of finding the same tumor type in both the bladder and the upper urinary tract [see Kurma, H., et al., Hinyokika Kyo 48(4), 199-202 (2002); and Miyake, H., et al., BJU Int. 85(1), 37-41 (2000)]. This finding makes it preferable for any radiotherapy to be administered systemically so that excretion through the kidneys will expose both the upper urinary tract and the urinary bladder to the agent. Intravesicular administration is likely to be blocked from reflux into the ureters, preventing any effect on the upper urinary tract. In addition, intravesicular administration of liquid sources of radionuclides into the bladder [Durrant, K. R., et al., J. Urol. 113(4), 480-502 (April 1975)] is complicated by the contamination risks associated with handling radioactivity in the surgical suite or clinical room of a hospital.

Conventional radiotherapy for urinary bladder cancer usually involves either a 3 field or 4 field, multi-session, external beam pelvic irradiation [see, for example, Muren, L. P., et al., Int. J. Radiat. Oncol. Biol. Pays. 50(3), 627637 (2001); Tsukamoto, S., et al., Scand J. Urol. Nephrol 36(5), 339-343 (2002); and Fokdal, L. et al., Acta Oncol. 43(8), 749-757 (2004)]. Therapy is complicated by the intra- and inter-fraction movements of the bladder, which is a mobile organ. The size, volume and position vary from one treatment to another, and even within one treatment, as urine collects in the bladder with movements of air and fecal volumes in the adjacent small and large bowels. Thus, it is difficult to direct radiation to a specific portion of the bladder that contains the tumor and broader fields are required. Recent developments in conformal therapy with intensity modulation radiation therapy and on-board imaging of the bladder prior to each treatment session can remove some of these uncertainties, but the problems associated with exposing large volumes of normal tissue in the path of the beams on the way to the bladder wall and out of beam dose to the remainder of the body are predicted to be associated with a risk of fatal secondary malignancies of 2-5% (see Kry S. F., et al., Int. J Radiat Oncol. Biol. Phys. 62(4), 1195-203 (Jul. 15, 2005)].

Previous studies have used a Y-90 colloidal solution for the treatment of bladder tumors [Durrant K R, Laing A H., J Urol. 113(4), 480-21 (April 1975)]. According to this article approximately 80 mL containing 100 mCi of the Y-90 solution was infused directly into the bladder delivering approximately 15 Gy to the urinary bladder. However, the handling and potential contamination issues associated with delivery of the isotope via a urethral catheter into the bladder made this process dangerous and impractical.

Morbidity was common after radical radiotherapy with bladder symptoms (requiring surgery in 6.3% of patients) and rectal symptoms (requiring surgery in 2.3% of patients) being most frequent. Recurrence of cancer occurred in over 40% of patients [see Chahal, R. et al., Euro. Urol. 3(3), 246-257 (2003)], and about 20% of patients who were initially treated with radiotherapy subsequently required cystectomy [see Chahal, Ri et al., Euro. Urol. 43(3), 246-257 (2003)]. Mortality in patients treated with radical radiotherapy was 0.3% at 30 days, 1.65% at 3 months, and 62.4% at 5 years.

Thus, there is a clear need for an improved, non-surgical therapy for the treatment of urinary bladder cancer.

The kidney is the organ in the body with primary responsibility for the removal of fluid and dissolved waste products in the blood. There are two primary mechanisms to accomplish these purposes. One is a passive mechanism called glomerular filtration. This occurs in the glomerulus of the kidneys which can filter molecules up to about 70,000 Daltons. In addition, there is an active transport mechanism called tubular secretion that uses energy to remove solutes from the body. The tubules can also reabsorb dissolved compounds back into the bloodstream.

Radiopharmaceuticals are drugs that have been designed to incorporate a radioisotope and to diagnose or treat disease. In some cases, the radionuclide without a carrier molecule is used, such as strontium-89 (Sr-89) for the treatment of pain associated with bone cancer. In other cases, the radionuclide is attached to a carrier molecule that directs the conjugate to the intended site. Examples of this type of radiopharmaceutical are the monoclonal antibodies specific to certain cancer tissues that have been labeled with isotopes having properties to both diagnose and treat cancer patients. In the case of diagnosis, the radioactive component of the radiopharmaceutical is a gamma emitter. The gamma photon has enough energy to be detected outside the body after administration. For example, technetium-99m (Tc-99m) radiopharmaceuticals are commercially available to diagnose a variety of disease states. Since Tc-99m is a pure gamma emitter it delivers a low dose rate to tissues in the patient. The gamma emission of 140 KeV allows for detection outside the body using a gamma camera Different technetium compounds are being used to deliver technetium to different tissues and attain diagnostic information about normal and abnormal functions or sites in the patient. Examples include phosphonic acid compounds of technetium for detecting bone tumors, cationic complexes of technetium for evaluating heart function, and aminocarboxylic acid complexes of technetium to evaluate renal function. However, in the case of treatment of disease being desired, various other radioisotopes have been chosen depending on the depth and the length of treatment desired.

A variety of radiopharmaceuticals have been designed to study kidney function. These renal agents are injected I.V. and are removed from the blood into the bladder by the kidneys. One use is to obtain information about the morphology of the kidneys. Examples of these include Tc-99m-glucoheptanate [Arnold, R. W., et al., J. Nucl. Med. 1A 357-367 (1975)] and Tc-99m-dimercaptosuccinc acid [Lin, M. S., et al., J. Nucl. Med. A534-35 (1974)]. The key features of these agents are their specificity and retention in the kidneys. Radiopharmaceuticals with short mean transit times in the renal system are desired to reduce radiation dose to the tubules and avoid localization in other issues.

Radiopharmaceuticals have also been used to study the glomerular filtration rate of the kidneys. The most popular agent for this evaluation has been Tc-99m-DTPA [Klopper, J. F., et al., J. Nucl. Med. 13, 107-110 (1972)]. The popularity of this agent is due to the ideal nuclear properties of Tc-99m plus the specificity for elimination of the agent by glomerular filtration with no tubular secretion or reabsorption.

For many years, the standard for measuring tubular secretion by the kidneys has been the use of I-131-orthoiodohippuran [TubisTubis, M., et al., Proc. Soc. Exp. Biol. Med. 103, 497 (1960)]. This agent was used because it mimics the clearance of p-aminohippurate, known to be secreted by the tubules in the kidneys. However, this agent has now been replaced by the Tc-99m-MAG₃ (MAG₃ means N-[N-[N-[(benzoylthio)acetyl]glycyl]glycyl]glycine) because of the better imaging properties of Tc-99m.

Radiopharmaceuticals are also used to treat disease. Typically particle emitters are used since they allow local energy deposition to selected target volumes. Beta-emitting radionuclides such as iodine-131 (I-131), samarium-153 (Sm-153), holmium-166 (Ho-166), yttrium-90 (Y-90), and lutetium-177 (Lu-177) have been used for the treatment of cancer. In addition, alpha emitters such as astatine-211 (At-211), actinium-225 (Ac-225), bismuth-212 (Bi-212), and bismuth-213 (Bi-213) have been proposed for use in disease treatment. In cases where the radionuclide is a metal ion, chelating agents are used to control the metal and deliver them to the site of the cancer. For example, U.S. Pat. No. 4,898,724 teaches the treatment of bone cancer using a phosphonic acid complexed to a radioactive beta emitter. In this case about one half of the radioactivity is deposited in the bone and the other half is excreted via the kidneys. Similar phosphonic acid chelates are taught in U.S. Pat. No. 4,882,142. A preferred embodiment of U.S. Pat. No. 4,882,142 is the complex formed between Ho-166 and DOTMP, a macrocyclic aminophosphonic acid. Clinical trials with this agent have been associated with significant renal toxicity. In addition U.S. Pat. No. 5,652,361 teaches the delivery of radionuclides to soft tissue tumor using a conjugate composed of a radionuclide attached to an antibody using a bifunctional chelating agent. The chelating agents used in U.S. Pat. No. 5,652,361 are aminocarboxylic acid derivatives derived from the macrocyclic amine, cyclen.

Another use of chelates has been as magnetic resonance contrast agents. WO 1986/002352 teaches the use of aminocarboxylic derivatives of macrocyclic amines chelated to the lanthanide metal ion gadolinium (Gd) as a contrast agent. A preferred embodiment of WO 1986/002352 is the chelate between Gd⁺³ and DOTA (i.e., 1,4,7,10-tetraazacyclododecane-1,4,7,10-tetraacetic acid). Gd-DTPA (i.e., diethylenetriamine-pentaacetic acid) has also been used commercially as a contrast agent (Magnevist® by Schering AG). These aminomethylenecarboxylic acid chelates are used as general blood pool agents. There is no localization in any organ and the chelates are known to be rapidly cleared by the kidneys into the bladder.

### SUMMARY OF THE INVENTION

This present new treatment for urinary bladder cancer would ideally treat both the bladder and the upper urinary tract. The therapy would be limited in depth of treatment to avoid damage to the gastrointestinal tract and the pelvic nerve plexuses. The therapy would ideally require limited numbers of treatments for initial therapy.

This invention provides a method to treat urinary bladder cancer patients with ionizing radiation that can specifically irradiate the lining of the bladder wall and is associated with minimal complications. This treatment can be is accomplished by delivering radionuclides to the bladder volume. An embodiment of this invention concerns a method of delivering of a compound of Formula (I)

R-L (I)

where:
R is a radionuclide; and
L is a ligand that is an organic molecule that binds the radionuclide,

which comprises administrating a therapeutically effective amount of such compound of Formula (I) to the urinary bladder by intravenous administration in such a patient or animal in need of such treatment where such compound is cleared from the blood via the kidneys and is deposited in the bladder.

The radiation emitted from these isotopes delivers a prescribed ablative radiation dose to the lining of the bladder wall. In addition, these agents are administered intravenously to allow exposure of the renal pelvis and ureters to the ionizing radiation which provides treatment of cancer cells that may have refluxed from the bladder into the ureters.

As a further embodiment, this invention includes the use of such a diagnostic agent first, followed by treatment with a radionuclide as discussed herein.

### DETAILED DESCRIPTION OF THE INVENTION

Currently all known radionuclide therapy methods are limited by marrow toxicity, including I-131, which is the most successful therapy, that is limited to 200 mCi.

This invention provides a method of administering a radionuclide designed to ablate cancer cells that grow in and spread along the bladder wall of a patient or animal that has been diagnosed with early stage bladder cancer. This treatment can be accomplished by delivering a therapeutically-effective amount of a radionuclide of Formula (I) to the urine in the bladder by an intravenous (IV) injection of a radioactive agent that is cleared via the kidneys into the bladder. In contrast to prior methods, the aim of this invention is to administer small volumes (1-5 mL) of the radiopharmaceutical solution intravenously. The I.V. administration of small volumes of radiopharmaceuticals is safe and routinely performed.

The compounds of this invention have the following formula:

R-L (I)

where:
R is a radionuclide; and
L is a ligand that is an organic molecule that binds the radionuclide,

which comprises administrating a therapeutically effective amount of such compound of Formula (I) to the urinary bladder by intravenous administration in such a patient or animal in need of such treatment where such compound is cleared from the blood via the kidneys and is deposited in the bladder.

L is a ligand that is an organic molecule that binds the radionuclide and is capable of transporting the radionuclide into the bladder via the kidneys after intravenous administration with a molecular weight small enough to clear the kidneys via either glomerular filtration or tubular transport.

If R is a metallic radionuclide then L is a chelating agent capable of binding said metallic radionuclide such that the complex remains stable in the blood until it is cleared from the blood by the kidneys. If R is a halogen, then L is an organic molecule capable of covalently binding R and remaining stable in the blood until L-R is cleared from the blood by the kidneys. Preferred molecular weight of L is less than 70,000 Daltons, more preferred is less than 1,000 Daltons.

The radionuclides, R in Formula (I), useful for this invention are particle-emitters such as alpha- or beta-emitters and low energy gamma-emitters. Selecting the optimum radionuclide for bladder cancer treatment depends on a number of factors related to the depth and thickness of cancer cells in the wall, the half-life of the isotope and availability for use in the clinic. Preferred radionuclides are beta-emitters with a half-life less than 2 weeks; more preferred are beta-emitters with energy greater than 1 MeV and half-lives less than 1 week; most preferred are beta-emitters with energy greater than 1.5 MeV and half-lives less than 3 days.

The higher energy beta-emitters allow for tissue penetration at depths up to 1 to 5 mm. The shorter half-life isotopes maximize the number of radioactive disintegrations during the bladder holding period. This could allow for multiple treatments, and will provide a larger safety margin to the patient and clinicians handling the dose. A short half-life is also useful for reducing the severity of potential spills and issues with contamination.

Radionuclides, R of Formula (I), useful for this invention are particle-emitters such as alpha- or beta-emitters or low energy gamma-emitters. An example of suitable radionuclides are the rare earth-type metal ions that have suitable radioisotopes, including La, Ce, Pr, Nd, Pm, Sm, Eu, Gd, Th, Dy, Ho, Er, Tm, Yb, Lu, Y and Sc; especially preferred are Sm, Ho, Lu, and Y. Preferred radioactive rare earth-type metal ions include Sm-153, Ho-166, Y-90, Pm-149, Gd-159, La-140, Lu-177, Yb-175, Sc47, and Pr-142; especially preferred are Sm-153, Ho-166, Y-90, and Lu-177. Other radioactive metal ions which are of interest for the purpose of this invention are Re-186, Re-188, Ru-97, Rh-105, Pd-109, Pt-197, Cu-67, Au-198, Au-199, Ga-67, Ga-68, In-111, In-113m, In-155m, Sn-117m, Pb-212, Bi-212, Bi-213, Ac-225, I-131 and At-211. Re-188 is a preferred isotope for use in this invention because it has (a) a short half-life, (b) an energetic beta; (c) a low abundant gamma photon that can be used to determine the biolocalization of the isotope, and (d) ready availability via a generator system from Tungsten-188 (W-188). In addition, Re-188 and Tc-99m as the MAG₃ chelate are known to be quickly removed from the body by tubular active transport. Another preferred isotope is Ho-166 because it (a) is easily produced in high yields in a nuclear reactor and (b) has ideal nuclear properties for use in this invention. Its high energy beta emissions are ideal for therapy and its low abundant low energy gamma emissions can be used for imaging. In addition, this radionuclide can be complexed with a variety of aminocarboxylic acid chelating agents that will clear rapidly via the kidneys into the bladder.

When the radioactive atom is a metal ion, complexes of the metal ions with chelating agents, L in Formula (I), can be used. Chelating agents of this invention include, but are not limited to, nitrilotriacetic acid (NTA), ethylenediaminetetraacetic acid (EDTA), hydroxyethylethylenediaminetriacetic acid (HEDTA), diethylenetriaminepentaacetic acid (DTPA), trans-1,2-diaminocyclohexanetetraacetic acid (CDTA), and 1,4,7,10-tetraazacyclododecanetetraacetic acid (DOTA). One such preferred complex is Re-188-MAG₃ (N-[N-[N-[(benzoylthio) acetyl]glycyl]glycyl]glycine) due to the rapid clearance properties of this chelate. In the case of I-131, compounds of this invention include small iodinated molecules that are readily cleared through the kidneys such as orthoiodohippuran.

In Formula (I) the ligand to metal ratio of the compound is from about 1:1 to about 100 to 1.

The present invention provides a simple patient specific approach to treating bladder cancer using a quantitative dosimetry approach. In the past, early stage bladder cancer has been treated with simple urethral instillation of a radioactive solution of a high energy beta emitting solution in the bladder and rinsing the bladder after a designated time selected to deliver an ablative dose to the bladder wall. This invention provides a quantitative approach to treatment of bladder cancer based on the pharmacokinetic data from a prior diagnostic workup. This present approach is used to determine the specific amount of a beta emitter that is required to deliver a therapeutic dose to the bladder wall. Thus the therapeutically-effective dose is determined for each patient. A microdosimeter may be used to determine when the bladder wall has received a prescribed radiation dose and the bladder may be emptied. Thus the bladder wall dose can be predicted from serial quantitative gamma camera images and the bladder wall dose can be measured using a simple microdosimeter placed in the bladder volume. A simple bladder wall dose measurement technique allows this procedure to be widely adopted in the urology clinic. This method allows for patient differences and cancer progress at time of treatment.

This present method allows for treatment of the entire bladder and upper urinary tract, with low volumes of radioactive material, with possible imaging, by I.V. route. These are each embodiments that benefit this method.

Thus, the compounds of Formula (I) comprise a radioisotope particle emitter, R, such as alpha- or beta-emitters or low energy gamma-emitters, associated with L, a chelating agent (for metallic radioisotopes) or other compound (by covalent bond for iodine or other non-metals) selected such that these permit delivery of the radionuclide by an I.V. injection that is then cleared into the bladder by the kidneys. Such treatment by radionuclides can be designed to either ablate the bladder wall or deliver a radiation dose to the bladder where the cancer is treated and the normal cells may then recover.

Furthermore, it is expected that these radioisotope compounds of Formula (I) are formulated with customary pharmaceutically-acceptable salts, adjuvants, binders, desiccants, diluents, and excipients. The route of administration may be any that allows for delivery of the radionuclide to be cleared into the bladder by the kidneys. Most preferred is I.V. administration. The radiopharmaceuticals of Formula (I) can be used by themselves or when desired in combination with chemotherapy or external beam therapy. When used in combination with other therapies, it is possible to reduce the amount of the other therapies and/or obtain better outcomes for the therapy. The present method for treatment of bladder cancer has superior target to non-target ratios compared with I-131 used for thyroid cancer treatment.

The invention will be further clarified by a consideration of the following examples, which are intended to be purely exemplary of the present invention.

### Example 1

### Preparation of Ho-166-DOTA

Ho-166 was prepared by neutron capture of Ho-165 at the University of Missouri Research Reactor. It was supplied as the solid nitrate in a plastic vial. The vial contained 3 mg of Ho-165. To this vial, 1 mL of 0.1M nitric acid was added to dissolve the salt. The activity of the sample was about 9 mCi. A volume of 30 μL of the Ho-166 solution was added to a solution containing 9.3 mg of DOTA (1,4,7,10-tetraazacyclododecane-1,4,7,10-tetraacetic acid) in 480 μL of water. The pH of the solution was adjusted with NaOH until the pH was about 10. It was then adjusted to 6.5 using HCl. The percent Ho-166 existing as a DOTA complex was determined by using cation exchange chromatography. The fraction of activity that is eluted from the column with saline with two 5 mL elutions is defined to be the % of the activity as a complex. By this method, greater than 97% of the Ho-166 was found to be complexed.

### Example 2

### Rat Biodistribution of Ho-166-DOTA

A volume of 30 μL of the solution of Example 1 was administered to Sprague Dawley Rats. The rats were placed in cages and sacrificed at 30, 60 and 120 minutes after injection. The organs and tissues of the rat were collected and compared to standards. Measurement of radioactivity was done with a T1-drifted NaI detector coupled to a multichannel analyzer. The results showed the % of the dose in the blood at 30, 60 and 120 minutes to be 7.0, 2.4 and 0.33 percent, respectively. The percent of the injected dose in the urine at 30 minutes was 61%. By 60 minutes 87% of the dose was in the urine. The amount of the injected dose found in the kidneys was 1.87% at 30 minutes and down to 0.5% of the injected dose at 2 hours. No appreciable amount of activity was found in any other organ. This data is consistent with the radioactivity being cleared quickly from the blood into the urinary bladder.

### Example 3

### Radiation Dose Estimates for Ho-166-DOTA

Dosimetry estimates for Ho-166-DOTA were made using the biodistribution data of Ho-166-DOTA in rats. The dose calculated to the bladder is very similar to that calculated using the nuclear decay properties of Ho-166 and using human blood clearance data from Tc-99m-DTPA studies. Since both Tc-99m DTPA and Ho-166-DOTA are cleared from the plasma through the kidneys into the bladder by the same mechanism, this is a legitimate, scientific assumption that allows for a good estimation of the doses to humans due to the intravenous (IV) administration of Ho-166-DOTA.

Radiation dose calculations were performed using the PC internal dosimetry program OLINDA distributed by Mike Stabin of Vanderbilt University. The residence times for Ho-166-DOTA for various organs in the rat model were found to be similar to human data for Tc-99m-DTPA. Table 1 below indicates the range of estimated radiation absorbed dose that would be delivered to the bladder wall and kidneys due to the administration of Ho-166-DOTA. The data was calculated based on the assumption that the bladder would be voided at 1, 2, or 3.5 hours. The table shows the estimated absorbed dose in rems per mCi of Ho-166 administered I.V.

TABLE 1
Radiation dose estimates to the bladder
wall and kidneys for Ho-166 DOTA

| Voiding | rem/mCi | rem/mCi |
|---|---|---|
| Interval | Bladder wall | Kidneys |
| 1 hr | 3.06 | 0.36 |
| 2 hr | 6.26 | 0.36 |
| 3.5 hr | 11.1 | 0.36 |

These results for Ho-166-DOTA are surprising and encouraging as a tumoricidal dose of 18 Gy can be delivered with about 300 mCi Ho-166 (assuming a voiding interval of 2 hours) without giving a significant dose to the kidneys. Finally, manipulation of the voiding pattern in the individual patient can be used to maximize the radiation dose to the bladder wall compared with the kidney. For example, voiding at 60 minutes results in a dose of 3.06 rem/mCi to the bladder wall, whereas voiding at 2 hours results in 6.26 rem/mCi.

These radiation dose estimates can be measured in real time either by quantitative serial imaging of the clearance of Ho-166-DOTA in the whole body with a pin hole camera, or by inserting a miniature dosimeter such as a microMOSFET into the urinary bladder and tracking the integrated dose delivered to the urine volume. Both methods can signal the stop time at which the prescribed dose of 18 Gy is delivered, so the patient can empty their bladder.

Although the invention and method have been described with reference to its preferred embodiments, those of ordinary skill in the art may, upon reading this disclosure, appreciate changes and modifications which may be made which do not depart from the scope and spirit of this invention as described above or claimed hereafter.

1. A method of delivering of a compound of Formula (I)
R-L (I)
where:
R is a radionuclide; and
L is a ligand that is an organic molecule that binds the radionuclide,
which comprises administrating a therapeutically effective amount of such compound of Formula (I) to the urinary bladder by intravenous administration in such a patient or animal in need of such treatment where such compound is cleared from the blood via the kidneys and is deposited in the bladder.

2. The method of claim 1 wherein R is a radionuclide that contains a beta-emitting isotope.

3. The method of claim 1 wherein R is a radionuclide that contains an alpha-emitting isotope.

4. The method of claim 1 wherein R is a radionuclide that contains a low energy gamma-emitting isotope.

5. The method of claim 1 wherein R is a radionuclide selected from the group consisting of Sm-153, Ho-166, Y-90, Pm-149, Gd-159, La-140, Lu-177, Yb-175, Sc-47, Pr-142, Re-186, Re-188, Ru-97, Rh-105, Pd-109, Pt-197, Cu-67, Au-198, Au-199, Ga-67, Ga68, In-111, In-113m, In-155m, Sn-117m, Pb-212, Bi-212, Bi-213, Ac-225, I-131 and At-211.

6. The method of claim 5 where R is a radionuclide selected from the group consisting of Ho-166, Y-90, Lu-177, Sm-153, Gd-159, Re-186 and Re-188.

7. The method of claim 6 where R is a radionuclide selected from the group consisting of Ho-166, Y-90, Lu-177, Sm-153 and Gd-159.

8. The method of claim 1 wherein the ligand, L, is an aminomethylenecarboxylic acid.

9. The method of claim 8 wherein the ligand is NTA, EDTA, DTPA, HEDTA, CDTA, DOTA, and derivatives thereof.

10. The method of claim 1 where the ligand, L, is in molar excess over the radionuclide, R.

11. The method of claim 10 where the ligand to radionuclide ratio of the compound is from about 1:1 to about 100 to 1.

12. The method of claim 1 where the molecular weight of L is less than 70,000 Daltons.

13. The method of claim 12 where the molecular weight of L is less than 1,000 Daltons.

14. A method of claim 1 wherein the kidney function of a patient is evaluated using a renal agent such as Tc-99m-DTPA or Tc-99m-MAG₃ prior to the administration of the therapeutic agent.

15. The method of claim 1 where the radiopharmaceutical is Re-188-MAG₃ and the patient is first evaluated using Tc-99m-MAG₃.

16. The method of claim 10 or 11 wherein the patient is treated with Ho-166-DOTA.

17. The method of claim 1 or 10 wherein the patient is treated with Re-188-MAG₃.

18. The method of claim 5 wherein the radionuclide is I-131.

19. The method of claim 18 wherein the radioactive complex is I-131-orthoiodohippuran.

20. The method of claim 1 wherein the radiopharmaceutical is administered in combination with external beam radiation and/or a chemotherapeutic agent.

21. The method of treatment of claim 1 wherein the patient is instructed not to urinate for a period time.

22. A method of claim 21 wherein the time period is 2 hours or greater.

23. A formulation comprising a compound of claim 1 formulated with customary pharmaceutically-acceptable salts, adjuvants, binders, desiccants, diluents, and excipients.

24. The formulation of claim 21 wherein a radionuclide, R, is a radioactive metal mixed with no less than an equal molar amount of a chelating agent, L, in acidic media and the pH is raised above 7 then brought back to about 7 prior to injection.

25. The formulation of claim 22 or 23 wherein the formulation contains calcium or magnesium.
